(19) United States
(12) Patent Application Publication
(10) Pub. No.: US 2026/0272519 A1
(43) Pub. Date: Sep. 17, 2026

(54) ORTHOPEDIC INTRAMEDULLARY NAIL SYSTEM FOR GREATER TROCHANTER FIXATION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Jordan Rakes, Cordova, TN (US); Charles R. Bennett, Memphis, TN (US); Paul Tornetta, III, Chestnut Hill, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/167,041

(22) PCT Filed: May 7, 2024
(86) PCT No.: PCT/US2024/028103

§ 371 Date: Sep. 19, 2025

Related U.S. Application Data
(60) Provisional application No. 63/466,415, filed on May 15, 2023.

(51) Int. Cl. A61B 17/72; A61B 17/74; A61B 17/80; A61B 17/86

(52) U.S. Cl. CPC A61B 17/72; A61B 17/744; A61B 17/8061; A61B 17/86

(57) ABSTRACT

An intramedullary (“IM”) nail system, and corresponding method, for fractured fixation of a patient's greater trochanter is disclosed. In some examples, the IM nail system includes an IM nail and a plate or washer. In use, the plate or washer includes one or more holes or openings for enabling a bone screw to pass through the plate or washer to engage a corresponding hole formed in the IM nail. Thus arranged, in use, the IM nail system facilitates fixation of the patient's greater trochanter by securing and/or compressing the patient's greater trochanter between the plate or washer and the IM nail.

### CROSS-REFERENCE TO RELATED APPLICATIONS

This is a non-provisional of, and claims the benefit of the filing date of, U.S. provisional patent application number 63/466,415, filed May 15, 2023, entitled “Orthopedic Intramedullary Nail System for Greater Trochanter Fixation,” the entirety of which application is incorporated by reference herein.

### FIELD OF THE DISCLOSURE

The present disclosure is directed to orthopedic implants (e.g., intramedullary (“IM”) nails) for stabilizing one or more patient's bones, bone portions, bone fragments, etc., and more specifically to an IM nail system arranged and configured to facilitate fixation of a greater trochanter fracture.

### BACKGROUND

Orthopedic fixation devices (implants) may be used, for example, to stabilize an injury, to support a bone fracture, to fuse a joint, and/or to correct a deformity. Orthopedic fixation devices may be attached permanently or temporarily, and may be attached to the bone at various locations, including implanted within a canal or other cavity of the bone, implanted beneath soft tissue and attached to an exterior surface of the bone, or disposed externally and attached by fasteners such as screws, pins, and/or wires. Some orthopedic fixation devices allow the position and/or orientation of two or more bone pieces, or two or more bones, to be adjusted relative to one another. Orthopedic fixation devices are generally machined or molded from isotropic materials, such as metals including, for example, titanium, titanium alloys, stainless steel, cobalt-chromium alloys, and tantalum.

An intramedullary (“IM”) nail is one type of orthopedic fixation device. The primary function of the IM nail is to stabilize the fracture fragments, and thereby enable load transfer across the fracture site while maintaining anatomical alignment of the bone. Currently, there are a large number of different commercially available IM nails in the marketplace.

One disadvantage of IM nails is that during fracture fixation there may be fractured fragments, which cannot be captured using the IM nail and screw offerings. This may be the result of limited space within the IM nail for screw holes or bone fragments, which may be too small or too soft to be captured by the available screws. For example, during intertrochanteric fractures treated with a cephalomedullary nail, greater trochanter fragments may not be adequately captured using the IM nail alone. Left untreated, greater trochanter fragments can result in bursitis or abductor mechanism deficiency.

One known solution for treating fractures of the greater trochanter fragments in intertrochanteric cases includes the use of additional cables and/or additional bone plates to capture the greater trochanter fragments. However, placing additional cables and/or additional bone plates increases the time and complexity of the surgery resulting in increased amounts of time under anesthesia, which may be deemed a greater risk than leaving the greater trochanter fragments unfixed.

Thus, there remains a need to provide improved orthopedic IM nail systems for internal fixation of a bone, and specifically, to treat greater trochanter fractures. The present disclosure satisfies this need and provides other benefits and advantages in a novel and unobvious manner.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

An intramedullary (“IM”) nail system for fractured fixation of a patient's greater trochanter is disclosed. In some examples, the IM nail system includes an IM nail and a bone plate or washer (terms bone plate and washer are used interchangeably herein without the intent to limit or distinguish). In use, the washer includes one or more holes or openings for enabling a bone screw to pass through the washer to engage a corresponding hole formed in the IM nail. In this manner, the washer is coupled to the IM nail via the bones screw. In some examples, placement of the washer and the bone screw though the washer and into the IM nail is guided by a targeting jig. Thus arranged, in use, the IM nail system facilitates fixation of the patient's greater trochanter by securing and/or compressing the patient's greater trochanter fractured fragment between the washer and the IM nail.

In some examples, an intramedullary (“IM”) nail system arranged and configured to facilitate greater trochanter fracture fixation is disclosed. The IM nail system including an IM nail, a washer, and a bone screw. The IM nail including a body, a proximal end portion, a distal end portion, and a plurality of screw openings. The washer including an outer surface, a bone contacting surface, and at least one opening extending between the outer surface and the bone contacting surface. In use, with the IM nail implanted within an intramedullary canal of a patient's femur and with the bone contacting surface of the washer positioned against the patient's greater trochanter, the bone screw is inserted through the opening formed in the washer and into one of the plurality of screw openings formed in the IM nail.

In any preceding or subsequent example, the bone screw compresses the greater trochanter between the washer and the IM nail.

In any preceding or subsequent example, the one of the plurality of screw openings formed in the IM nail is threaded, the threaded screw opening arranged and configure to engage the threads of the bone screw to compress the greater trochanter between the washer and the IM nail.

In any preceding or subsequent example, the IM nail system further includes a targeting jig arranged and configured to be coupled to the proximal end portion of the IM nail, the targeting jig arranged and configured to facilitate alignment of the at least one opening formed in the washer with the one of the plurality of screw openings formed in the IM nail.

In any preceding or subsequent example, the washer includes a central region including a plurality of arms extending therefrom, the central region including the at least one opening.

In any preceding or subsequent example, the at least one opening formed in the washer includes first and second openings.

In any preceding or subsequent example, the bone contacting surface of the washer includes a plurality of spikes extending therefrom.

In any preceding or subsequent example, the washer includes a plurality of suture openings formed therein.

In some examples, a method of fixing a patient's greater trochanter is also disclosed. The method including inserting an IM nail into an intramedullary canal of a patient's femur; positioning a washer adjacent the patient's greater trochanter; and inserting a bone screw through an opening formed in the washer and into a screw opening formed in the IM nail.

In any preceding or subsequent example, inserting the bone screw compresses the patient's greater trochanter between the washer and the IM nail.

In any preceding or subsequent example, the screw opening formed in the IM nail is threaded.

In any preceding or subsequent example, positioning the washer includes coupling a targeting jig to the IM nail and utilizing the targeting jig to align the opening in the washer with the screw openings formed in the IM nail.

Examples of the present disclosure provide numerous advantages. For example, the system and/or method enables fixation of a patient's greater trochanter using an IM nail. In use, by utilizing an IM nail, a washer, and a bone screw that passes through the washer and IM nail, a simple and efficient manner of securing and/or compressing the patient's greater trochanter fractured fragment is provided by eliminating the requirement to install multiple implants, which requires increased time under anesthesia and larger incisions.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1A is an anterior view of an example of an IM nail system including an IM nail, a washer, and a bone screw in accordance with one or more features of the present disclosure, the IM nail system shown implanted within a patient's bone;

FIG. 1B is a lateral view of the IM nail system shown in FIG. 1A;

FIG. 1C is a top view of the IM nail system shown in FIG. 1A;

FIG. 2A is a top view of an example of a washer in accordance with one or more features of the present disclosure, the washer forming part of the IM nail system shown in FIG. 1A;

FIG. 2B is a bottom view of the washer shown in FIG. 2A;

FIG. 2C is a side view of the washer shown in FIG. 2A;

FIG. 3A is a top view of an alternate example of a washer in accordance with one or more features of the present disclosure, the washer forming part of the IM nail system shown in FIG. 1A;

FIG. 3B is a side view of the washer shown in FIG. 3A;

FIG. 4A is a detailed view of a proximal end portion of an IM nail, the IM nail forming part of the IM nail system shown in FIG. 1A;

FIG. 4B is a perspective view of a targeting jig coupled to the IM nail shown in FIG. 4A; and

FIG. 4C is an alternate perspective view of the targeting jig coupled to the IM nail shown in FIG. 4B.

It should be understood that the drawings are not necessarily to scale and that the disclosed examples are sometimes illustrated diagrammatically and in partial views. In certain instances, details which are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features or the like of an IM nail system, and corresponding method, for greater trochanter fracture fixation will now be described more fully hereinafter with reference to the accompanying drawings, in which one or more features of the IM nail system, and corresponding method, will be shown and described. It should be appreciated that the various features or the like may be used independently of, or in combination, with each other. It will be appreciated that an IM nail system and method as disclosed herein may be embodied in many different forms and should not be construed as being limited to the examples set forth herein. Rather, these examples are provided so that this disclosure will convey certain features of the IM nail system and method to those skilled in the art.

Disclosed herein are various IM nail systems, and corresponding methods, including one or more features arranged and configured to facilitate greater trochanter fracture fixation.

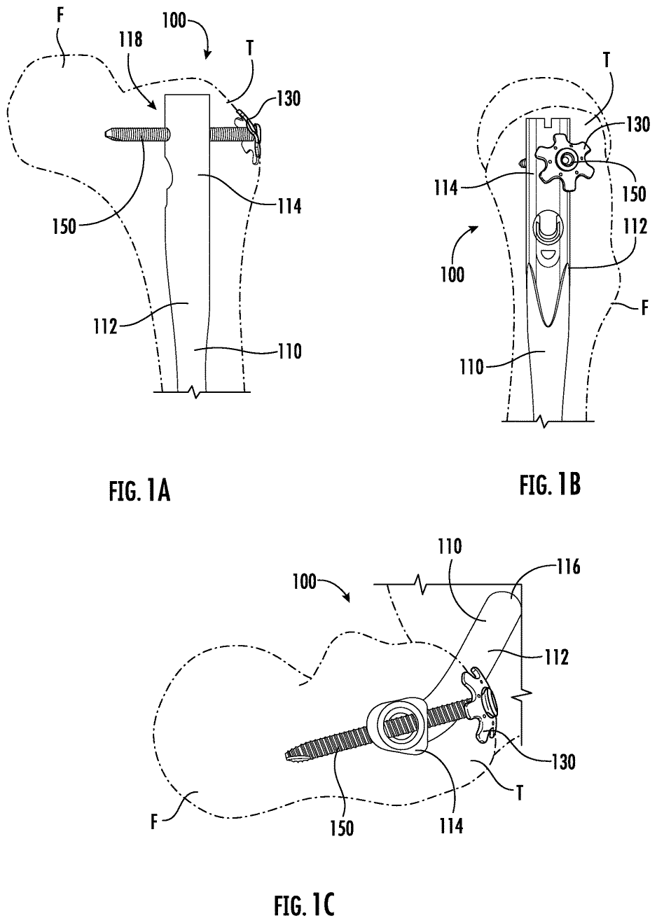

Referring to FIGS. 1A-1C, an example of an IM nail system 100 in accordance with one or more features of the present disclosure is illustrated. As illustrated, the IM nail system 100 includes an IM nail 110, a washer or bone plate 130, and at least one bone screw 150 (terms washer and bone plate used interchangeably herein without the intent to limit or distinguish).

In use, as will be readily appreciated by one of ordinary skill in the art, the IM nail 110 is arranged and configured to be implanted into the intramedullary canal of a patient's femur. As such, the IM nail 110 may also be referred to as a femoral nail. Generally speaking, the IM nail 110 includes a body 112 such as, for example, a cannulated body. The body 112 includes a proximal end portion 114 and a distal end portion 116. The proximal end portion 114 and the distal end portion 116 including a plurality of screw openings, holes, slots, etc. 118 (terms used interchangeably herein without the intent to limit) arranged and configured to receive a bone fastener, screw, etc. (terms used interchangeably herein without the intent to limit or distinguish). In some examples, the screw openings can be threaded. Alternatively, the screw openings can be non-threaded, or have any other configuration now known or hereafter developed. For reasons that will become apparent below, by providing a threaded screw opening, the IM nail 110 facilitates compression against the fractured fragment of the patient's greater trochanter T without relying on screw fixation with the patient's bone (e.g., femur F).

In use, the IM nail 110 may be any suitable femoral nail now known or hereafter developed. In addition, while the present disclosure will describe and illustrate an IM nail system, and corresponding method, for fracture fixation of a greater trochanter, one or more features of the present disclosure may have applicability to other parts of the patient's body. As such, the present disclosure should not be limited to any particular IM nail or part of the patient's body unless specifically claimed.

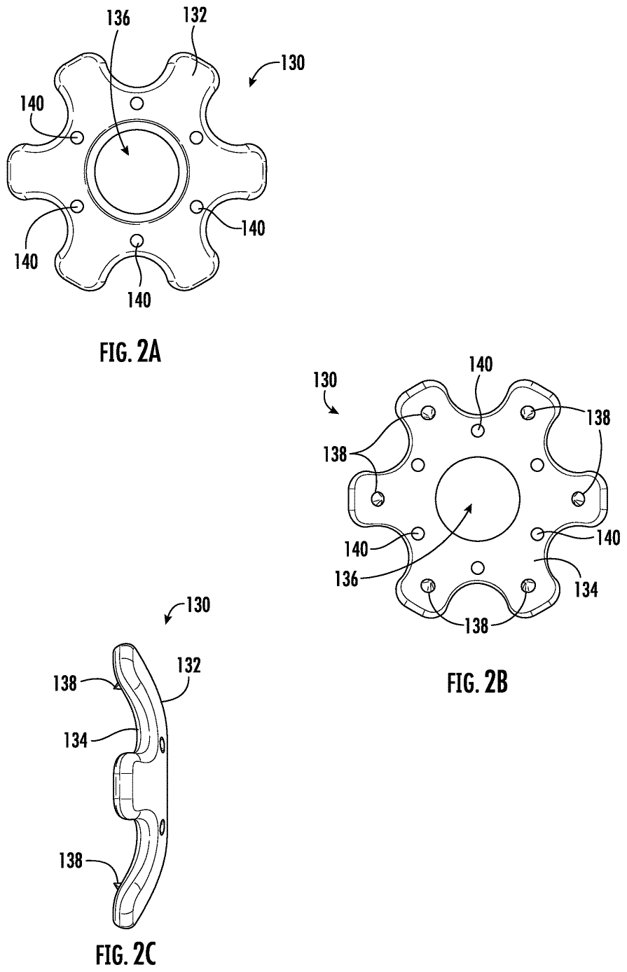

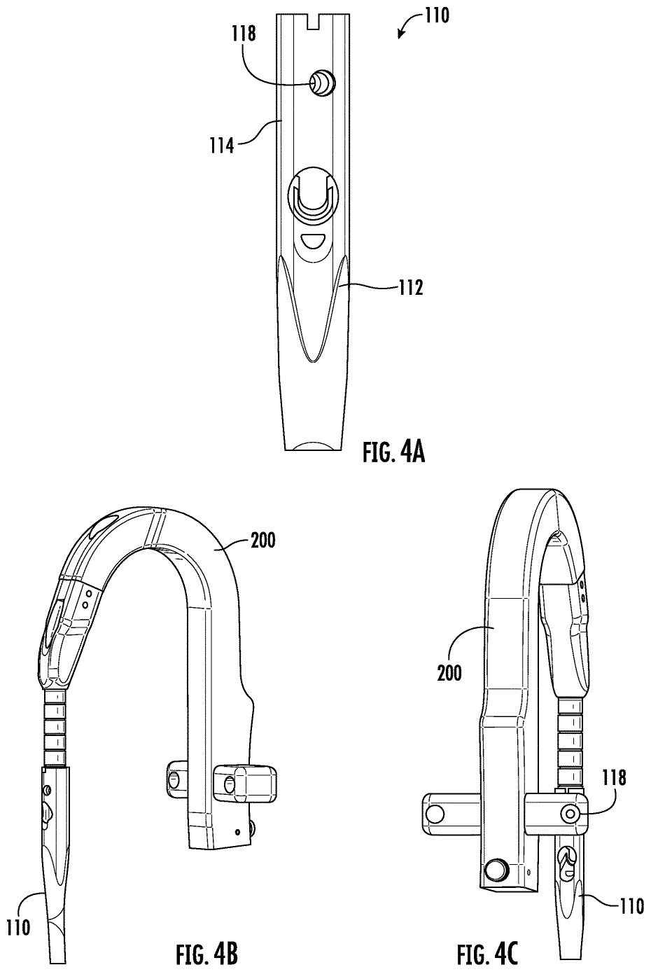

With reference to FIGS. 1A-1C, and with additional reference to FIGS. 2A-2C, the washer 130 includes a first or outer surface 132, a second or bone contacting surface 134, and a hole 136 extending between the first and second surfaces 132, 134. In use, with the IM nail 110 implanted within the intramedullary canal of the patient's femur F and with the washer 130 positioned against the patient's greater trochanter T, the hole 136 formed in the washer 130 may be aligned with one of the plurality screw openings 118 formed in the proximal end portion 114 of the body 112 of the IM nail 110. For example, as illustrated in FIGS. 4A-4C, a targeting jig 200 may be coupled to the proximal end portion 114 of the body 112 of the IM nail 110. Thereafter, utilizing the targeting jig 200, as will be readily appreciated by one of ordinary skill in the art, the hole 136 formed in the washer 130 can be aligned with one of the plurality of screw openings 118 formed in the body 112 of the IM nail 110 so that a bone screw 150 can be inserted through the hole 136 formed in the washer 130 and into the screw opening 118 formed in the IM nail 110. Thus arranged, in use, a fracture of the greater trochanter T can be fixed by compressing the fractured fragment of the patient's greater trochanter T between the washer 130 and the IM nail 110 via the bone screw 150 (e.g., the greater trochanter fractured fragment can be fixed via implanting a targeted bone screw through the washer and IM nail to apply compression across the fragment to encourage healing).

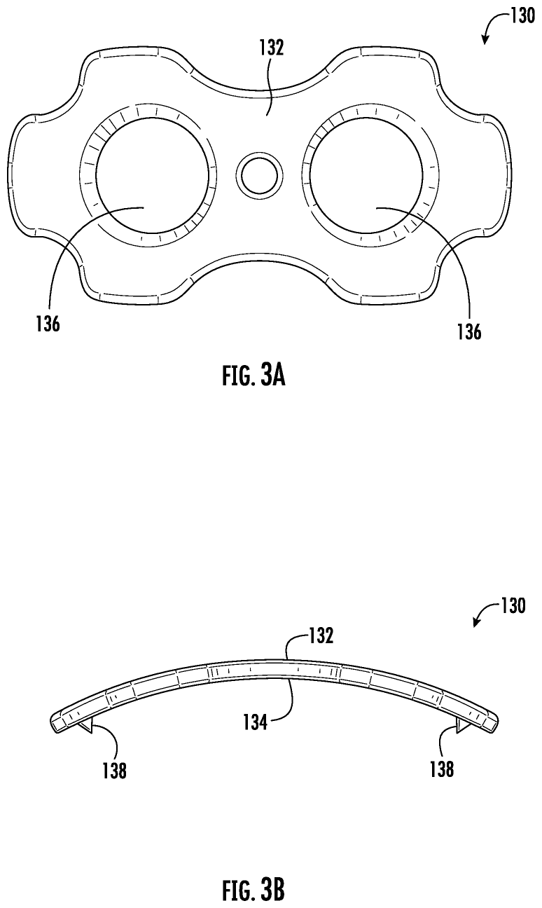

In some examples, as illustrated in FIGS. 2A-2C, the washer 130 may generally have the form of a circular plate with a plurality of arms extending therefrom. For example, as illustrated, the washer 130 may include a central region including a plurality of arms extending therefrom, the central region including the at least one hole 136. In use, the washer 130 may be arranged and configured to be malleable and/or flexible. For example, the plurality of arms may be flexible relevant to the central region so that the washer 130 can conform to the shape of the patient's bone (e.g., greater trochanter). However, this is but one configuration and the washer 130 may have any suitable shape and./or configuration now known or hereafter developed. For example, while the washer 130 has been illustrated in FIGS. 2A-2C with a single central opening, other configurations are envisioned. For example, with reference to FIGS. 3A and 3B, the washer 130 may include a plurality of screw holes 136 such as, for example, first and second screw openings. By providing multiple screw openings, surgeons have increased flexibility in placing the washer 130 onto the patient's greater trochanter T to accommodate different fracture patterns and patient anatomy.

In some examples, as shown, the second or bone facing surface 134 of the washer 130 may include a plurality of spikes 138 extending therefrom to facilitate increased contact (e.g., grip) with the patient's greater trochanter T.

In addition, and/or alternatively, in some examples, the washer 130 may include a plurality of suture openings 140 for reattaching soft tissue.

As previously mentioned, with reference to FIGS. 4A-4C, utilization of a targeting jig 200 and IM nail system 100 is shown with the jig 200 providing targeted trajectory through the IM nail 110. In use, the targeting jig 200 may be used to facilitate screw hole trajectory for the placement of the washer 130.

Thus arranged, in use, a surgeon can implant the IM nail 110 into the intramedullary canal of the patient's femur F. Thereafter, as appropriate, utilizing a targeting jig 200 or other now known or hereafter developed targeting mechanism, the surgeon can position a washer 130 onto the patient's greater trochanter T and insert a bone screw 150 through the washer 130 and into the IM nail 110 to secure and/or compress the fractured fragment of the patient's greater trochanter T. For example, in use, the IM nail 110 may include a threaded opening formed therein for receiving the bone screw 150. Thus arranged, by inserting the bone screw 150 through the opening 136 formed in the washer 130 and into the threaded opening formed in the IM nail 110, the greater trochanter and/or fracture can be compressed between the washer 130 and the IM nail 110.

The foregoing description has broad application. Accordingly, the discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The term “a” or “an” entity, as used herein, refers to one or more of that entity. As such, the terms “a” (or “an”), “one or more” and “at least one” can be used interchangeably herein. The use of “including,” “comprising,” or “having” and variations thereof herein is meant to encompass the items listed thereafter and equivalents thereof as well as additional items. Accordingly, the terms “including,” “comprising,” or “having” and variations thereof are open-ended expressions and can be used interchangeably herein. The phrases “at least one”, “one or more”, and “and/or”, as used herein, are open-ended expressions that are both conjunctive and disjunctive in operation.

All directional references (e.g., proximal, distal, upper, underside, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order, and relative sizes reflected in the drawings attached hereto may vary.

What is claimed is:

1. An intramedullary (“IM”) nail system arranged and configured to facilitate greater trochanter fracture fixation, the IM nail system comprising:
an IM nail including a body, a proximal end portion, a distal end portion, and a plurality of screw openings;
a washer including an outer surface, a bone contacting surface, and at least one opening extending between the outer surface and the bone contacting surface; and
a bone screw;
wherein, in use, with the IM nail implanted within an intramedullary canal of a patient's femur and with the bone contacting surface of the washer positioned against the patient's greater trochanter, the bone screw is inserted through the opening formed in the washer and into one of the plurality of screw openings formed in the IM nail.

2. The IM nail system of claim 1, wherein the bone screw compresses the greater trochanter between the washer and the IM nail.

3. The IM nail system of claim 1, wherein the one of the plurality of screw openings formed in the IM nail is threaded, the threaded screw opening arranged and configure to engage the threads of the bone screw to compress the greater trochanter between the washer and the IM nail.

4. The IM nail system of claim 1, further comprising a targeting jig arranged and configured to be coupled to the proximal end portion of the IM nail, the targeting jig arranged and configured to facilitate alignment of the at least one opening formed in the washer with the one of the plurality of screw openings formed in the IM nail.

5. The IM nail system of claim 1, wherein the washer comprises a central region including a plurality of arms extending therefrom, the central region including the at least one opening.

6. The IM nail system of claim 1, wherein the at least one opening formed in the washer comprises first and second openings.

7. The IM nail system of claim 1, wherein the bone contacting surface of the washer includes a plurality of spikes extending therefrom.

8. The IM nail system of claim 1, wherein the washer includes a plurality of suture openings formed therein.

9. A method of fixing a patient's greater trochanter, the method comprising:
inserting an IM nail into an intramedullary canal of a patient's femur;
positioning a washer adjacent the patient's greater trochanter; and
inserting a bone screw through an opening formed in the washer and into a screw opening formed in the IM nail.

10. The method of claim 9, wherein inserting the bone screw compresses the patient's greater trochanter between the washer and the IM nail.

11. The method of claim 10, wherein the screw opening formed in the IM nail is threaded.

12. The method of claim 9, wherein positioning the washer comprises coupling a targeting jig to the IM nail and utilizing the targeting jig to align the opening in the washer with the screw openings formed in the IM nail.
